(19) United States
(12) Patent Application Publication
(10) Pub. No.: US 2026/0210936 A1
(43) Pub. Date: Jul. 23, 2026

(54) Method for Determining Water Cut and Oil - Gas Ratio in a Three - Phase System

(71) Applicants: China University of Petroleum (Beijing), Houston, TX (US); STenergy LLC, Houston, TX (US)

(72) Inventors: Hui Li, Beijing (CN); Quanjun Wang, Cypress, TX (US); Ben Li, Beijing (CN)

(21) Appl. No.: 19/356,132

(22) Filed: Oct. 12, 2025

(30) Foreign Application Priority Data
Jan. 23, 2025 (CN) 202510107315

(51) Int. Cl. G01N 33/28

(52) U.S. Cl. CPC G01N 33/2823; G01N 33/2841; G01N 33/2847

(57) ABSTRACT

The present application provides a method for determining water cut and oil-gas ratio in a three-phase system. The method comprises: acquiring the total mass, total volume, and gas volume of produced fluids at the wellhead of a target well within a preset monitoring period, wherein the produced fluids are in an oil-water-gas three-phase mixed state; obtaining the emulsion ratio, emulsion volume coefficient, oil density, water density, and gas density of the fluids; determining liquid-phase mass and liquid-phase volume of the fluids according to the total mass, total volume, gas volume, and gas density; calculating the oil mass fraction in the liquid phase of the fluids based on the liquid-phase mass, liquid-phase volume, emulsion ratio, emulsion volume coefficient, oil density, and water density; and determining water cut and oil-gas ratio according to the oil mass fraction in the liquid phase. This solution addresses the problem of low accuracy in conventional calculations of water cut and oil-gas ratio, thereby achieving efficient and accurate determination.

### TECHNICAL FIELD

The present application relates to the technical field of oil and gas production, and in particular to a method for determining water cut and oil-gas ratio in a three-phase system.

### BACKGROUND OF THE INVENTION

During reservoir development and petroleum production, complex phenomena often occur in which oil, gas, and water three-phase fluids coexist in the wellbore and surface facilities, accompanied by emulsification or partial emulsification between oil and water.

At present, surface separators or test manifolds separate the produced fluids into a gas phase and a liquid phase (oil+water) for measurement. The gas phase is generally measured by volumetric meters or flow meters combined with density conversion to obtain gas production. The liquid phase is usually estimated for water cut by means of an online water content analyzer or offline sampling and laboratory testing, and then oil and water production rates are inferred by combining the volume or flow rate. However, when the liquid-phase oil-water ratio or properties are complex and dissolved gas or free gas exists, this method produces relatively high measurement errors. Moreover, after emulsification of oil and water, the total volume is not a simple linear addition, which inevitably results in incomplete phase separation and separation delay, leading to calculation deviations.

Oil-water emulsification is a multiphase micro-dispersion phenomenon jointly caused by colloidal asphaltenes in crude oil, natural surfactants or added chemical agents, and shear forces in pipelines. When part of the water is encapsulated in crude oil to form an emulsion, molecular structure and intermolecular interactions may cause actual volume expansion or contraction. In engineering calculations or daily metering, in order to simplify the calculation of emulsification states, it is generally assumed that there is no emulsification (treating oil volume and water volume as directly additive), or that complete emulsification occurs (all water is emulsified by oil). However, for cases of “partial emulsification” or “stagewise emulsification” (only part of the water is encapsulated by oil), water cut and oil-gas ratio cannot be accurately determined.

With respect to the technical problem of accurately determining water cut and oil-gas ratio in the case of three-phase fluids with partial emulsification, no effective solution has been proposed so far.

### SUMMARY OF THE INVENTION

The purpose of the present application is to provide a method for determining water cut and oil-gas ratio in a three-phase system, which can accurately and efficiently determine the water cut and oil-gas ratio of three-phase fluids.

The present application provides a method for determining water cut and oil-gas ratio in a three-phase system, which is implemented as follows:

A method for determining water cut and oil-gas ratio in a three-phase system, the method comprising:
acquiring the total mass, total volume, and gas volume of fluids produced at the wellhead of a target well within a preset monitoring period, wherein the produced fluids are in an oil-water-gas three-phase mixed state;
obtaining the emulsion ratio, emulsion volume coefficient, oil density, water density, and gas density of the fluids;
determining the liquid-phase mass and liquid-phase volume of the fluids according to the total mass, total volume, gas volume, and gas density;
calculating the oil mass fraction in the liquid phase of the fluids based on the liquid-phase mass, liquid-phase volume, emulsion ratio, emulsion volume coefficient, oil density, and water density;
determining the water cut and oil-gas ratio of the fluids according to the oil mass fraction in the liquid phase.

In one embodiment, calculating the oil mass fraction in the liquid phase of the fluids based on the liquid-phase mass, liquid-phase volume, emulsion ratio, emulsion volume coefficient, oil density, and water density comprises:
calculating the oil mass fraction in the liquid phase of the fluids according to the following formula:

$${V}_{l} = ( 1 - r ) \frac{( 1 - α ) {M}_{l}}{{ρ}_{w}} + δ [ \frac{α {M}_{l}}{{ρ}_{0}} + r \frac{( 1 - α ) {M}_{l}}{{ρ}_{w}} ]$$

wherein α represents the oil mass fraction, r represents the emulsion ratio, δ represents the emulsion volume coefficient, Mₗ represents the liquid-phase mass, ρ_{w} represents the water density, ρₒ represents the oil density, and Vₗ represents the liquid-phase volume.

In one embodiment, calculating the oil mass fraction in the liquid phase of the fluids based on the liquid-phase mass, liquid-phase volume, emulsion ratio, emulsion volume coefficient, oil density, and water density comprises:
calculating the oil mass fraction in the liquid phase of the fluids according to the following formula:

$${V}_{l} = ( 1 - r ) \frac{( 1 - α ) {M}_{l}}{{ρ}_{w}} + ( 1 - s ) \frac{α {M}_{l}}{{ρ}_{0}} δ [ s \frac{α {M}_{l}}{{ρ}_{0}} + r \frac{( 1 - α ) {M}_{l}}{{ρ}_{w}} ]$$

wherein α represents the oil mass fraction, r represents the emulsion ratio, δ represents the emulsion volume coefficient, Mₗ represents the liquid-phase mass, ρ_{w} represents the water density, ρₒ represents the oil density, Vₗ represents the liquid-phase volume, and s represents the proportion of the oil phase that can be emulsified.

In one embodiment, determining the water cut of the fluids according to the oil mass fraction in the liquid phase of the fluids comprises:
calculating the water cut according to the following formula:

$${f}_{w} = \frac{( 1 - α ) {ρ}_{o}}{α {ρ}_{w} + ( 1 - α ) {ρ}_{o}}$$

wherein f_{w} represents the water cut, α represents the oil mass fraction, ρ_{w} represents the water density, and ρₒ represents the oil density.

In one embodiment, determining the oil-gas ratio of the fluids according to the oil mass fraction in the liquid phase of the fluids comprises:
calculating the oil-gas ratio according to the following formula:

$$GOR = \frac{{v}_{g} {ρ}_{o}}{α ( {M}_{t o t a l} - {ρ}_{g} {v}_{g} )}$$

wherein GOR represents the oil-gas ratio, ν_{g} represents the gas volume, ρ_{w} represents the water density, ρₒ represents the oil density, and Mₜₒₜₐₗ represents the total mass.

In one embodiment, after determining the oil-gas ratio of the fluids according to the oil mass fraction in the liquid phase of the fluids, the method further comprises:
converting the gas volume into a standard volume under standard temperature and pressure;
calculating the oil-gas ratio under standard conditions according to the following formula:

$${GOR}_{s t d} = \frac{{V}_{g}^{s t d}}{{V}_{o}} \\ wherein {V}_{g}^{s t d}$$

represents the standard volume under standard temperature and pressure, and Vₒ represents the oil phase volume.

In one embodiment, obtaining the emulsion ratio, emulsion volume coefficient, oil density, water density, and gas density of the fluids comprises:
acquiring the temperature and pressure at the wellhead of the target well within a preset monitoring period;
retrieving a pre-established data reference table, in which the emulsion ratio, emulsion volume coefficient, oil density, water density, and gas density of the fluids of the target well under different temperatures and pressures, as determined by external experiments, are recorded;
identifying from the data reference table the emulsion ratio, emulsion volume coefficient, oil density, water density, and gas density corresponding to the acquired temperature and pressure;
taking the identified emulsion ratio, emulsion volume coefficient, oil density, water density, and gas density as those of the fluids.

In one embodiment, after determining the water cut and oil-gas ratio of the fluids according to the oil mass fraction in the liquid phase of the fluids, the method further comprises:
determining the required separator pressure and the required injection amount of emulsifier according to the water cut and oil-gas ratio;
adjusting the separator pressure according to the determined required separator pressure;
injecting the emulsifier according to the determined required injection amount.

An electronic device comprising a processor and a memory for storing instructions executable by the processor, wherein when executed by the processor, the instructions perform the steps of the above method.

A computer-readable storage medium having stored thereon a computer program or instructions, wherein when executed by a processor, the computer program or instructions perform the steps of the above method.

The method for determining water cut and oil-gas ratio in a three-phase system provided in the present application combines the total mass, total volume, and gas volume of fluids produced at the wellhead of a target well within a preset monitoring period, together with the obtained emulsion ratio, emulsion volume coefficient, oil density, water density, and gas density of the fluids, to calculate the oil mass fraction in the liquid phase of the fluids, thereby further determining the water cut and oil-gas ratio. That is, by introducing the emulsion ratio and emulsion volume coefficient into the calculation of water cut and oil-gas ratio, the present application solves the technical problem of low accuracy in existing calculations of water cut and oil-gas ratio and achieves the technical effect of efficiently and accurately determining water cut and oil-gas ratio.

### DESCRIPTION OF THE DRAWINGS

In order to more clearly illustrate the technical solutions of the embodiments of the present application or of the prior art, the drawings required for describing the embodiments or the prior art are briefly introduced below. It is apparent that the drawings described below merely represent some embodiments recorded in the present application. For those of ordinary skill in the art, other drawings may also be obtained based on these drawings without creative efforts.

FIG. 1 is a flowchart of a method for determining water cut and oil-gas ratio in a three-phase system according to one embodiment of the present application.

FIG. 2 is a flowchart of a method for automatically calculating water cut and oil-gas ratio according to one embodiment of the present application.

FIG. 3 is a block diagram of a hardware structure of an electronic device for determining water cut and oil-gas ratio in a three-phase system according to one embodiment of the present application.

FIG. 4 is a schematic diagram of a module structure of an apparatus for determining water cut and oil-gas ratio in a three-phase system according to one embodiment of the present application.

### SPECIFIC EMBODIMENTS

In order to enable those skilled in the art to better understand the technical solutions of the present application, the embodiments of the present application will be clearly and completely described below in conjunction with the accompanying drawings. It is obvious that the described embodiments are only a part of the embodiments of the present application, rather than all of the embodiments. Based on the embodiments of the present application, all other embodiments obtained by those skilled in the art without creative efforts shall fall within the protection scope of the present application.

It should also be noted that, in the embodiments of the present specification, certain software, components, models, and other existing industry solutions may be mentioned, which should be regarded as exemplary. The purpose is only to illustrate the feasibility of implementing the technical solutions of the present application, but it does not mean that the applicant has used or necessarily will use such solutions.

Considering the coexistence of oil, gas, and water three-phase fluids, when the gas production is relatively large, if the oil-water distribution is inferred only based on the liquid mass, significant deviations may occur. For example, for some heavy oil wells with relatively high gas density, ignoring the gas mass will overestimate the mass of the liquid phase, thereby affecting the accuracy of water cut and gas-oil ratio (GOR). Separator parameters and empirical curves generally only enable volumetric separation, for example, calculating the gas phase volume based on the gas production of a separator and then regarding the remaining mass as the liquid phase. This method produces relatively poor accuracy in cases with severe emulsification. In addition, the transition of dissolved gas in the oil phase into free gas also leads to inaccurate estimation of oil phase volume. That is, existing GOR calculations are generally based on the assumption that oil volume is known. However, when the ratio of oil to water in the liquid phase is unclear or part of the water is emulsified in oil, the oil volume cannot be accurately obtained. Ignoring emulsification or ignoring the effect of free gas will cause the GOR result to deviate from reality.

Furthermore, the existing water cut estimation methods only target oil-water two-phase fluids (ignoring the gas phase) or treat the gas phase merely as a volumetric deduction, without sufficient correction for the mass of gas. That is, in existing metering processes, only the presence or absence of emulsification is distinguished, while key parameters such as the amount of water emulsified and the volumetric deviation caused by emulsification are not considered, thereby resulting in low accuracy of calculated water cut and GOR.

To this end, the present embodiment provides a three-phase model that not only fully considers the influence of the gas phase on the total mass but also finely distinguishes partially emulsified water from free water layering. By introducing the emulsion ratio (that is, the proportion of water being emulsified) and the emulsion volume coefficient (that is, quantifying the effect of emulsion expansion or contraction) into the liquid phase, and combining mass and volume conservation equations, the ratio of oil and water phases is obtained. Based on this, the water cut can be calculated simultaneously, and the gas-oil ratio (GOR) can be derived from the oil phase volume, thereby achieving one-stop and accurate three-phase metering.

In other words, to address the problem of low accuracy in calculating water cut and oil-gas ratio in complex three-phase fluid scenarios, especially under significant emulsification effects, the present embodiment proposes a solution that, under the premise of known total quantities, gas phase information, emulsion ratio, and emulsion volume coefficient, simultaneously calculates water cut and oil-gas ratio. This method enables accurate determination of water cut and oil-gas ratio, thereby making three-phase metering and reservoir analysis more accurate and scientific.

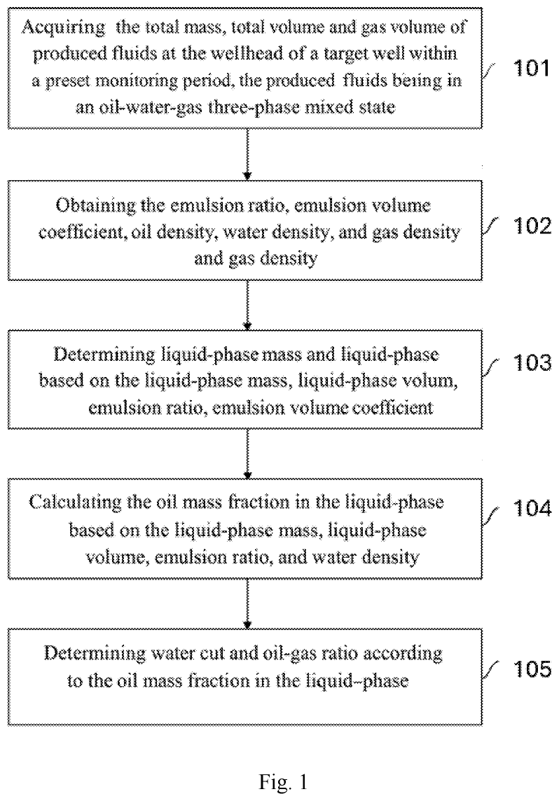

FIG. 1 shows a flowchart of a method for determining water cut and oil-gas ratio in a three-phase system according to one embodiment of the present application. Although the present application provides the method operation steps or apparatus structures as shown in the embodiments or figures below, additional or fewer operation steps or module units may be included in the method or apparatus based on routine or non-creative efforts. For steps or structures without necessary causal relationships in logic, the execution order of the steps or the module structures of the apparatus is not limited to the execution order or module structures described in the embodiments of the present application or shown in the figures. When the methods or module structures are applied in practical apparatuses or terminal products, they may be executed sequentially or in parallel (for example, in parallel processors, multithreaded environments, or even distributed processing environments).

Specifically, as shown in FIG. 1, the above method for determining water cut and oil-gas ratio in a three-phase system may comprise the following steps:
Step 101: acquiring the total mass, total volume, and gas volume of fluids produced at the wellhead of a target well within a preset monitoring period, wherein the produced fluids are in an oil-water-gas three-phase mixed state;
Step 102: obtaining the emulsion ratio, emulsion volume coefficient, oil density, water density, and gas density of the fluids;
Step 103: determining the liquid-phase mass and liquid-phase volume of the fluids according to the total mass, total volume, gas volume, and gas density;
Step 104: calculating the oil mass fraction in the liquid phase of the fluids based on the liquid-phase mass, liquid-phase volume, emulsion ratio, emulsion volume coefficient, oil density, and water density;

Specifically, the liquid-phase mass and liquid-phase volume may first be determined according to the total mass, total volume, gas volume, and gas density of the fluids, and then the liquid-phase mass may be determined according to the total mass of the fluids, the gas volume, and the gas density, using the following formula:

$${M}_{l} = {M}_{t o t a l} - {ρ}_{g} {v}_{g}$$

wherein Mₗ represents the liquid-phase mass, Mₜₒₜₐₗ represents the total mass, ν_{g} represents the gas volume, and ρ_{g} represents the gas density;
further determining the liquid-phase volume according to the total volume and the gas volume by the following formula:

$${V}_{l} = {V}_{t o t a l} - {v}_{g}$$

wherein Vₗ represents the liquid-phase volume, and Vₜₒₜₐₗ represents the total volume.

Further, the oil mass fraction in the liquid phase of the fluids is calculated based on the liquid-phase mass, liquid-phase volume, emulsion ratio, emulsion volume coefficient, oil density, and water density.

For the oil mass fraction, two calculation methods are provided in this embodiment:
Method 1: calculating the oil mass fraction in the liquid phase of the fluids according to the following formula:

$${V}_{l} = ( 1 - r ) \frac{( 1 - α ) {M}_{l}}{{ρ}_{w}} + δ [ \frac{α {M}_{l}}{{ρ}_{o}} + r \frac{( 1 - α ) {M}_{l}}{{ρ}_{w}} ]$$

wherein α represents the oil mass fraction, r represents the emulsion ratio, δ represents the emulsion volume coefficient, Mₗ represents the liquid-phase mass, ρ_{w} represents the water density, ρₒ represents the oil density, and νₗ represents the liquid-phase volume.

Method 2: introducing the “oil emulsification ratio s,” wherein s represents the proportion of the oil phase that can be emulsified, and then calculating the oil mass fraction in the liquid phase of the fluids according to the following formula:

$${V}_{l} = ( 1 - r ) \frac{( 1 - α ) {M}_{l}}{{ρ}_{w}} + ( 1 - s ) \frac{α {M}_{l}}{{ρ}_{o}} δ [ s \frac{α {M}_{l}}{{ρ}_{o}} + r \frac{( 1 - α ) {M}_{l}}{{ρ}_{w}} ]$$

wherein α represents the oil mass fraction, r represents the emulsion ratio, δ represents the emulsion volume coefficient, Mₗ represents the liquid-phase mass, ρ_{w} represents the water density, ρₒ represents the oil density, νₗ represents the liquid-phase volume, and s represents the proportion of the oil phase that can be emulsified.

Step 105: determining the water cut and oil-gas ratio of the fluids according to the oil mass fraction in the liquid phase of the fluids.

In implementation, it is considered that the determination of the water cut of the fluids may be performed either from the perspective of mass ratio or from the perspective of volume ratio:

For example, determining the oil-gas ratio from the perspective of mass ratio according to the following formula:

$$GOR = \frac{{v}_{g} {ρ}_{o}}{α ( {M}_{t o t a l} - {ρ}_{g} {v}_{g} )}$$

wherein GOR represents the oil-gas ratio, ν_{g} represents the gas volume, ρ_{w} represents the water density, ρₒ represents the oil density, and Mₜₒₜₐₗ represents the total mass.

For example, determining the oil-gas ratio from the perspective of volume ratio according to the following method:
converting the gas volume into a standard volume under standard temperature and pressure, and calculating the oil-gas ratio under standard conditions according to the following formula:

$${GOR}_{s t d} = \frac{{V}_{g}^{s t d}}{{V}_{o}} \\ wherein {V}_{g}^{s t d}$$

represents the standard volume under standard temperature and pressure, and Vₒ represents the oil phase volume.

For the water cut, the water cut may be calculated according to the oil mass fraction in the liquid phase of the fluids by the following formula:

$${f}_{w} = \frac{( 1 - α ) {ρ}_{o}}{α {ρ}_{w} + ( 1 - α ) {ρ}_{o}}$$

wherein f_{w} represents the water cut, α represents the oil mass fraction, ρ_{w} represents the water density, and ρₒ represents the oil density.

That is, by combining the total mass, total volume, and gas volume of fluids produced at the wellhead of a target well within a preset monitoring period, together with the obtained emulsion ratio, emulsion volume coefficient, oil density, water density, and gas density of the fluids, the oil mass fraction in the liquid phase of the fluids is calculated, thereby further determining the water cut and oil-gas ratio of the fluids. In other words, by introducing the emulsion ratio and emulsion volume coefficient of the fluids into the calculation of water cut and oil-gas ratio, the technical problem of low accuracy in existing calculations of water cut and oil-gas ratio is solved, and the technical effect of efficiently and accurately determining water cut and oil-gas ratio is achieved.

In implementation, the emulsion ratio, emulsion volume coefficient, oil density, water density, and gas density of the fluids may be determined through external experiments or environmental simulation. Specifically, the emulsion ratio, emulsion volume coefficient, oil density, water density, and gas density of the fluids of the target well under different temperatures and pressures may be recorded. Then, during actual calculation, the temperature and pressure at the wellhead of the target well within a preset monitoring period may be acquired; a pre-established data reference table may be retrieved, in which the emulsion ratio, emulsion volume coefficient, oil density, water density, and gas density of the fluids of the target well under different temperatures and pressures, as determined by external experiments, are recorded; the emulsion ratio, emulsion volume coefficient, oil density, water density, and gas density corresponding to the acquired temperature and pressure may be identified from the data reference table; and the identified emulsion ratio, emulsion volume coefficient, oil density, water density, and gas density may be taken as those of the fluids.

Furthermore, by the above method, the water cut and oil-gas ratio of the fluids may be determined according to the oil mass fraction in the liquid phase of the fluids. After determining the water cut and oil-gas ratio, the required separator pressure and the required injection amount of emulsifier may be determined according to the water cut and oil-gas ratio. Then, the separator pressure may be adjusted according to the determined required separator pressure, and the emulsifier may be injected according to the determined required injection amount.

Based on the method for determining water cut and oil-gas ratio in a three-phase system described above, the present embodiment further provides a three-phase fluid processing system applied to oilfield sites, comprising:
1) sensor equipment, disposed at the wellhead of the target well or at the inlet and outlet of each separator, configured to measure in real time the temperature, pressure, total mass, total volume, and gas volume of fluids produced at the wellhead of the target well or at each separator within a preset monitoring period;
2) a memory, configured to store a pre-established data reference table, in which the emulsion ratio, emulsion volume coefficient, oil density, water density, and gas density of the fluids of the target well under different temperatures and pressures, as determined by external experiments, are recorded;
3) a control processor, configured to acquire temperature, pressure, total mass, total volume, and gas volume from the sensor equipment, and to obtain the emulsion ratio, emulsion volume coefficient, oil density, water density, and gas density of the fluids corresponding to the acquired temperature and pressure; and further configured to calculate the oil mass fraction in the liquid phase of the fluids based on the total mass, total volume, gas volume, gas density, oil density, water density, emulsion ratio, and emulsion volume coefficient of the fluids, and to calculate and output the water cut and oil-gas ratio of the fluids according to the oil mass fraction in the liquid phase.
4) a visual human-machine interaction device, configured to display in real time the determined water cut and oil-gas ratio of the fluids through an on-site operation terminal at the oil well or through a remote central control room interface.

Specifically, the above sensor equipment may comprise: a temperature detector, configured to acquire real-time temperature; and a pressure detector, configured to acquire real-time pressure. That is, temperature data and pressure data may be obtained through the sensor equipment.

Furthermore, the above sensor equipment may also comprise: a storage tank disposed at the wellhead of the target well or at the inlet and outlet of each separator; a weight sensor disposed below the storage tank, configured to detect weight data; and a gauge disposed in the storage tank, configured to sense the heights of water, oil, and gas in the storage tank so as to determine the total mass, total volume, and gas volume of the collected fluids. That is, by providing a weight sensor for weighing below the storage tank and a gauge in the storage tank to obtain the heights of various fluids in the tank, the volumes of the respective fluids may be determined.

After acquiring weight, temperature, pressure, and volume data through the sensor equipment, the data are transmitted to the control processor, which may directly obtain the water cut and oil-gas ratio. That is, in actual field use, it is only necessary to set a storage tank at the oil outlet, and then, by measuring the height, temperature, pressure, and mass data, the oil-gas ratio and water cut may be obtained.

To obtain the emulsion ratio and emulsion volume coefficient, stirring and shearing test equipment may be arranged in the three-phase fluid processing system applied to the oil well site. The stirring and shearing test equipment may be disposed in a bypass pipeline to simulate actual working conditions by changing shear intensity and temperature, so as to determine the actual expansion ratio or shrinkage ratio of the fluid volume, thereby determining the emulsion ratio and emulsion volume coefficient of the fluids, and storing them in the memory. The emulsion ratio and emulsion volume coefficient may be set as curve variation values corresponding to different pressures and temperatures.

To perform control under actual working conditions, the three-phase fluid processing system applied to the oil well site may further comprise central control equipment, configured to adjust the pressure of the separator and the injection amount of demulsifier according to the water cut and oil-gas ratio.

The above method will be described below in conjunction with a specific embodiment. However, it should be noted that this specific embodiment is only for better illustrating the present application and does not constitute an undue limitation of the present application.

In view of the problems in the prior art, in which water cut and GOR are determined in a stepwise manner, requiring separate oil-water and oil-gas separation measurements and thereby increasing equipment and operating costs, and in which the accuracy of the determined water cut and GOR is low because emulsification is not considered, the present embodiment provides, for commonly encountered oil-water-gas three-phase mixed fluids in oilfield well sites and surface gathering and transportation environments, a digital method and system for synchronously calculating water cut and oil-gas ratio with consideration of gas phase mass correction and partial oil-water emulsification effects. On the basis of conventional three-phase separation and metering, the method introduces “gas volume” and “gas density” to accurately deduct gas phase mass, and further adopts “emulsion ratio” and “emulsion volume coefficient” to quantitatively characterize the volumetric deviation and dispersion proportion of emulsified water, thereby achieving the following objectives:
1) accurately deducting the influence of the gas phase, avoiding miscalculation of gas mass into the liquid phase under conditions of high gas-oil ratio or significant dissolved gas, and reducing calculation errors of water cut and oil-gas ratio caused by gas density variation or measurement deviations;
2) flexibly handling partial emulsification phenomena by distinguishing “free water” and “emulsified water encapsulated in oil” through the setting of emulsion ratio r and emulsion volume coefficient δ, thereby overcoming the technical problem of inaccurate prediction results caused by existing assumptions of no emulsification or complete emulsification. Furthermore, in the present embodiment, water cut and oil-gas ratio are obtained synchronously, that is, both the water content in the liquid phase (water cut) and the volumetric ratio of the gas phase to the oil phase (GOR) can be obtained through a single calculation, thereby facilitating separator design, production metering, and reservoir dynamic analysis, and reducing the inconvenience caused by multiple devices or segmented manual operations in the prior art;
3) digitalization and online implementation. In the present embodiment, formulas and logical flows are embedded into the real-time data acquisition and transmission network of well sites or surface metering systems. By means of embedded computing units or host computer software, oil-water distribution, water cut, and oil-gas ratio are automatically solved, and data storage, visualization, and remote monitoring are supported, thereby meeting the requirements of digitalized and automated management in modern oilfields. In this way, various on-site conditions with high water cut, high viscosity, complex emulsification, and significant gas phase influence may be more comprehensively adapted, providing a simple, reliable metering method and its supporting system for oilfield production, produced fluid metering, separator optimization, and reservoir evaluation.

Specifically, the present embodiment provides a three-phase metering method that considers partial emulsification and can simultaneously calculate water cut and oil-gas ratio, comprising:
S1: measuring or obtaining the total mass Mₜₒₜₐₗ, total volume Vₜₒₜₐₗ, gas volume ν_{g}, and gas density ρ_{g} of the oil-water-gas three-phase system, and calculating the liquid-phase mass Mₗ=Mₜₒₜₐₗ−ρ_{g}ν_{g} and the liquid-phase volume Vₗ=Vₜₒₜₐₗ−ν_{g};
S2: determining the emulsion ratio r and the emulsion volume coefficient δ;
S3: determining the oil density ρₒ and the water density ρ_{w};
S4: calculating the oil mass fraction α in the liquid phase according to the following volume equation:

$${V}_{l} = ( 1 - r ) \frac{( 1 - α ) {M}_{l}}{{ρ}_{w}} + δ [ \frac{α {M}_{l}}{{ρ}_{o}} + r \frac{( 1 - α ) {M}_{l}}{{ρ}_{w}} ]$$

wherein r represents the emulsion ratio, and δ represents the emulsion volume coefficient.
S5: after calculating the oil mass fraction α, calculating the water cut according to the following formula:

$${f}_{w} = \frac{( 1 - α ) {ρ}_{o}}{α {ρ}_{w} + ( 1 - α ) {ρ}_{o}}$$

calculating the oil-gas ratio according to the following formula:

$$GOR = \frac{{v}_{g} {ρ}_{0}}{α ( {M}_{t o t a l} - {ρ}_{g} {v}_{g} )} ∘$$

In step S2 above, the emulsion ratio r and the emulsion volume coefficient δ may be determined as follows: placing the oil-water mixture to be tested (that is, the sample) into a variable-speed stirring or shearing device, then simulating the actual reservoir environment by adjusting the stirring time, temperature, and shear intensity, and further measuring the actual volume and phase separation of the reservoir system after emulsification, thereby obtaining the emulsion ratio r and the emulsion volume coefficient δ.

In step S2 above, the emulsion ratio r and the emulsion volume coefficient δ may also be determined as follows: employing an online sensor to monitor in real time the volume change of the mixed liquid, determining the emulsion volume coefficient δ based on the volume change of the mixed liquid, and further combining phase-separation sampling to measure the proportion of free water, thereby determining the emulsion ratio r according to the proportion of free water.

In step S3 above, the oil density ρₒ and the water density ρ_{w} may be determined as follows: simulating the reservoir system, temperature, and pressure conditions, and then measuring the oil density ρₒ and water density ρ_{w} using a laboratory densitometer or an online density meter. If the temperature and pressure of the produced fluids change, the oil density ρₒ and water density ρ_{w} are correspondingly converted according to known PVT rules, so as to ensure that they are under the same working conditions as the gas density ρ_{g} and other parameters.

In the present embodiment, considering adaptation to different gas states, the calculation of the gas-oil ratio (GOR) is extended. When GOR needs to be defined under standard conditions, the field-measured V_{g} may first be converted to standard temperature and pressure, and then substituted into the following formula to obtain the GOR under standard conditions, namely GOR_{std}:

$${GOR}_{s t d} = \frac{{V}_{g}^{s t d}}{{V}_{o}}$$

Furthermore, for the case where part of the gas is dissolved in the oil phase, the free gas volume V_{g} may be re-determined after deducting the amount of dissolved gas, and then the GOR may be calculated based on the re-determined ν_{g}, so that the calculation result is more consistent with the actual conditions of the on-site separator or process flow.

Considering that different emulsification conditions have different impacts on the results, in order to adapt to more application scenarios, in the present embodiment, for the volume conservation equation, in addition to introducing α, an “oil emulsification ratio s” is further introduced, wherein s represents the proportion of the oil phase that can be emulsified. Based on this, the volume conservation equation is extended as follows:

$${V}_{l} = ( 1 - r ) \frac{( 1 - α ) {M}_{l}}{{ρ}_{w}} + ( 1 - s ) \frac{α {M}_{l}}{{ρ}_{0}} δ [ s \frac{α {M}_{l}}{{ρ}_{o}} + r \frac{( 1 - α ) {M}_{l}}{{ρ}_{w}} ]$$

By means of such a multi-parameter coupled solving approach, the results can satisfy more complex emulsification scenarios. Likewise, after determining α, the water cut f_{w} and the GOR may be calculated according to the above method.

The above water cut f_{w} and GOR may be applied in automated metering systems at oilfield sites. By inputting the measured parameters ρ_{g}, ν_{g}, Mₜₒₜₐₗ, Vₜₒₜₐₗ, ρₒ, ρ_{w}, δ and r into an embedded computing unit, a is solved in real time, and then the water cut f_w and GOR are calculated and output. When it is detected that α∉[0,1] or that the value of α is excessively large or small, the system automatically alarms or triggers data correction.

Specifically, the above method may be linked with the operating status of demulsification equipment or multi-stage separators. According to the real-time calculated water cut f_{w} and GOR, the separator pressure or emulsifier injection amount may be automatically adjusted to optimize separation efficiency. For cases of ultra-high water cut or high gas-oil ratio, early warning may be issued and corresponding process adjustments may be adopted to improve oil-water and gas-liquid separation performance.

In the present embodiment, an oil-water-gas three-phase fluid metering apparatus is further provided, comprising: a gas-phase measuring unit, a liquid-phase mass detecting unit, and a computing control unit connected in sequence, wherein the gas-phase measuring unit is configured to acquire the gas volume ν_{g} and density ρ_{g} and transmit them to the computing control unit; the liquid-phase mass detecting unit is configured to combine with the total mass of the system to generate the liquid-phase mass Mₗ and provide parameters ρₒ, ρ_{w}, δ and r; and the computing control unit is configured to calculate and output the water cut and oil-gas ratio based on the above parameters, and to display or transmit the results to an upper-level system. When the calculated oil mass fraction α exceeds the allowable range or abnormal parameters are detected by the system, automatic alarm is triggered or the operating conditions of the separator are adjusted.

In the present embodiment, a digital system is further provided, comprising: a data acquisition module, a communication interface and real-time data transmission module, a computing and logic processing module, a database/storage module, and a visualization/human-machine interaction module. The data acquisition module is configured to acquire data of the three-phase system including total mass, gas-phase volume, gas density, oil-water density, emulsion ratio, and emulsion volume coefficient from oilfield sites, separator control systems, or downhole/wellhead sensor networks. The communication interface and real-time data transmission module support wired or wireless communication methods, specifically including but not limited to Ethernet, 5G/4G, industrial bus protocols, or satellite links, for continuously acquiring real-time data from sensors or downhole devices and transmitting the acquired data to the computing and logic processing module. The computing and logic processing module is embedded with calculation instructions and logic flows corresponding to the above calculation method, and is configured to automatically deduct gas-phase mass and volume according to the input real-time data, thereby calculating the total mass and total volume of the liquid phase; to establish and solve the partial emulsification volume conservation equation to obtain the oil mass fraction α; and to calculate the water cut and oil-gas ratio based on the solved α. If α is detected to be outside the normal range or emulsification parameters are mismatched, a warning is issued and data correction or process adjustment is triggered. The database/storage module is configured to store model parameters, real-time and historical measurement data, and result information including the calculated α, f_{w}, and GOR, and to support playback or comparative analysis. The visualization/human-machine interaction module is configured to display key results such as water cut and oil-gas ratio to users in real time through graphical interfaces, reports, or cloud interfaces, and to interact with external systems (for example, separator control units or upper-level monitoring platforms) for data exchange or control operations, thereby realizing digital management and online automated regulation of oilfield production.

The above oil-water-gas three-phase fluid processing system may comprise:

### 1) Wellsite/Separator Environment:

During the process in which fluids produced from the oil well are brought to the surface through the wellhead, after preliminary separation by a primary separator, the produced fluids still remain in an oil-water-gas three-phase mixed state, wherein part of the water is emulsified in oil. To this end, a digital metering system may be configured on the separator/gathering pipeline. The digital metering system may comprise: a sensor network configured to measure the total mass of the three-phase fluids (or to acquire the total flow rate and convert the total mass based on the total flow rate), the gas volume V_{g} (which may be obtained by a gas flow meter or volumeter), the gas density ρ_{g} (which may be obtained by an online densitometer or converted using an equation of state), and the oil density ρₒ and water density ρ_{w} (which may be obtained by two online densitometers respectively disposed at the oil and water outlets, or obtained through temperature and pressure correction). Furthermore, a stirring/shearing test unit may be provided to acquire the emulsion ratio r and the emulsion volume coefficient δ. To obtain more accurate emulsion ratio r and emulsion volume coefficient δ, bypass pipelines or laboratory simulation devices may be used to vary shear intensity, temperature, and other conditions in the test system so as to determine the actual volumetric expansion/shrinkage, thereby obtaining more accurate emulsion ratio r and emulsion volume coefficient δ, so as to obtain more reliable parameters.

### 2) the Digital System Comprises:

a data acquisition module and a communication interface, configured to acquire in real time information such as ν_{g}, Mₜₒₜₐₗ, Vₜₒₜₐₗ, ρₒ, ρ_{w}, δ and r from sensors or separator control systems, and to transmit the data to the computing and logic processing module via a 4G/5G network or industrial Ethernet;
The computing and logic processing module, configured to embed core formulas and solving algorithms, to establish a liquid-phase volume equation by automatically deducting gas-phase mass so as to solve for α, and then to calculate the water cut f_{w} and the gas-oil ratio (GOR) based on the calculated α;
the database/storage module, configured to store operating parameters, historical data, and calculation results;
the visualization/human-machine interaction module, configured to display calculation results in real time and to allow remote retrieval of historical data through a wellsite operation terminal or a remote central control room interface.

By way of a specific example, at an oilfield site, a gas flowmeter with temperature and pressure compensation and a gas densitometer are installed. The gas volume ν_{g} (m³/h level) and the gas density ρ_{g} (kg/m³) are acquired in real time through the gas flowmeter and the gas densitometer, and the acquired gas volume V_{g} (m³/h level) and the gas density ρ_{g} (kg/m³) are transmitted in real time to the communication module. Suppose that in the current acquisition cycle, the sampled data are ν_{g}=0.35 m³ and ρ_{g}=2.0 kg/m³.

A high-precision mass flowmeter or a weighing sensor is configured at the oilfield site to obtain the total mass Mₜₒₜₐₗ of the system; and the total volume Vₜₒₜₐₗ of the container may be determined through equipment calibration or comprehensive measurement. For example, Mₜₒₜₐₗ=1500 kg, Vₜₒₜₐₗ=2.0 m³. Under the same temperature and pressure conditions, the oil density and water density may be measured or corrected through PVT (Process Verification Test) to be ρₒ=850 kg/m³ and ρ_{w}=1000 kg/m³. Through a bypass pipeline or online sampling test, it is determined that under the current flow and temperature conditions, about 40% of the water enters the emulsion, and the measured volume expands by about 3%. Based on this, the emulsion ratio r=0.4 and the emulsion volume coefficient δ=1.03 may be determined.

Furthermore, under extreme temperature and pressure conditions, PVT correction may be performed for oil density, water density, and gas volume. For this purpose, if a multi-stage separation system is adopted at the oil well, gas-phase data and liquid-phase data may be measured at the inlet and outlet of each separator, so as to perform PVT correction on the data, thereby improving measurement accuracy.

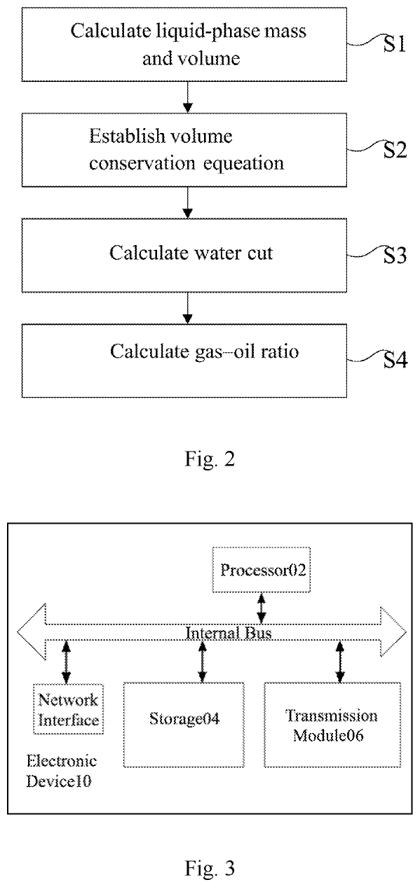

Based on the above obtained parameters, the water cut and gas-oil ratio may be automatically calculated according to the method shown in FIG. 2:
S1: calculating the liquid-phase mass and volume:

$${M}_{l} = {M}_{t o t a l} - {ρ}_{g} {v}_{g} = 1500 - ( 2 \times 0.35 ) = 1500 - 0.7 = 1499.3 kg$$

$${V}_{l} = {V}_{t o t a l} - {v}_{g} = 2 - 0.35 = 1.65 {m}^{3}$$

S2: establishing a volume conservation equation:

$${V}_{l} = ( 1 - r ) \frac{( 1 - α ) {M}_{l}}{{ρ}_{w}} + δ [ \frac{α {M}_{l}}{{ρ}_{o}} + r \frac{( 1 - α ) {M}_{l}}{{ρ}_{w}} ]$$

Let α=Mₒ/Mₗ, and by substituting the above values item by item, the linear solver in the computing and logic processing module automatically calculates the oil mass fraction α=0.68, indicating that oil accounts for about 68% of the liquid-phase mass.
S3: calculating the water cut f_{w}:

$${f}_{w} = \frac{( 1 - α ) {ρ}_{o}}{α {ρ}_{w} + ( 1 - α ) {ρ}_{o}} = \frac{( 1 - 0.68 ) \times 850}{0.68 \times 1000 + 0.32 \times 850} \approx 0.28$$

S4: calculating the gas-oil ratio (GOR):
oil phase volume:

$${V}_{o} = \frac{α {M}_{l}}{{ρ}_{o}} \approx \frac{0.68 \times 1499.3}{850} \approx 1.2 {m}^{3}$$

gas-oil ratio (GOR):

$$GOR = \frac{{v}_{g}}{{V}_{o}} = \frac{0.35}{1.2} \approx 0.29$$

Furthermore, if it is necessary to determine the GOR under standard conditions, the gas volume may be converted to standard conditions and then the ratio may be calculated to determine the GOR under standard conditions.

For the above digital system, the results may be displayed and automatically fed back. Specifically, the computing and logic processing module may transmit the calculated f_{w}=0.28 and GOR=0.29 (dimensionless or in m³/m³ form) to the database and display them in real time on the interface of the visualization module. If parameters such as ρ_{g} or ν_{g} change in the next measurement cycle, the system automatically recalculates and updates the results. If, during the detection process, it is identified that a [0,1] or that the value of a is excessively large or excessively small, the system may issue a warning prompt and suggest that field engineers check the operating conditions of the sensors or separators, clean the storage tank, and then recalculate f_{w} and GOR.

In the above example, sensor data and calculation results may be transmitted back to the central control room of the oilfield or to a cloud platform through 4G/5G or industrial Ethernet. For the central control room, separator pressure, demulsifier injection amount, or production allocation may be adjusted according to the real-time distribution of water cut and GOR of each wellsite/separator, thereby improving separation efficiency and system stability. In this way, centralized determination of water cut and GOR is realized, avoiding the cumbersome process in the prior art of separately estimating water cut and then manually calculating gas-oil ratio. Furthermore, this approach more closely matches actual three-phase partial emulsification conditions, which improves the accuracy of determined water cut, especially in wells with high water cut, high viscosity, and strong emulsification tendency, thereby reducing metering errors. This enables fine control of the difference between dissolved gas and free gas, effectively capturing the dynamic variation of gas-oil ratio, which has significant value for pipeline transportation and reservoir evaluation.

Through the above approach, water cut and gas-oil ratio may be accurately calculated under actual wellsite conditions, and the results may be digitally displayed and transmitted, thereby providing effective guidance for the oil and gas production process.

The method embodiments provided in the present application may be executed in a mobile terminal, a computer terminal, or a similar computing device. As an example of running on an electronic device, FIG. 3 is a block diagram of the hardware structure of an electronic device for determining water cut and oil-gas ratio in a three-phase system according to the present application. As shown in FIG. 3, the electronic device 10 may comprise one or more (only one is shown in the figure) processors 02 (the processor 02 may comprise, but is not limited to, processing devices such as a microcontroller unit (MCU) or a field-programmable gate array (FPGA)), a memory 04 for storing data, and a transmission module 06 for communication functions. Those skilled in the art may understand that the structure shown in FIG. 3 is merely illustrative and does not limit the structure of the electronic device. For example, the electronic device 10 may further comprise more or fewer components than those shown in FIG. 3, or may have a configuration different from that shown in FIG. 3.

The memory 04 may be used for storing software programs and modules of application software, such as program instructions/modules corresponding to the method for determining water cut and oil-gas ratio in a three-phase system in the embodiments of the present application. By executing the software programs and modules stored in the memory 04, the processor 02 executes various functional applications and data processing, thereby implementing the above method for determining water cut and oil-gas ratio in a three-phase system. The memory 04 may comprise high-speed random access memory and may further comprise non-volatile memory, such as one or more magnetic storage devices, flash memory, or other non-volatile solid-state memory. In some instances, the memory 04 may further comprise memory remotely disposed relative to the processor 02, which remote memory may be connected to the electronic device 10 through a network. Examples of the above network include, but are not limited to, the Internet, intranets, local area networks, mobile communication networks, and combinations thereof.

The transmission module 06 is used to receive or transmit data via a network. Specific examples of the above network may include a wireless network provided by a communication service provider of the electronic device 10. In one example, the transmission module 06 comprises a network interface controller (NIC), which may be connected with other network devices through a base station to communicate with the Internet. In one example, the transmission module 06 may be a radio frequency (RF) module configured to communicate with the Internet wirelessly.

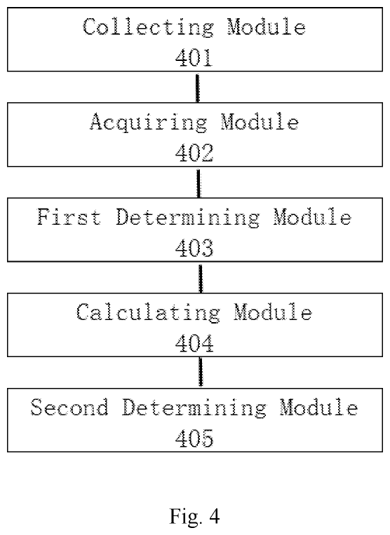

At the software level, the apparatus for determining water cut and oil-gas ratio in a three-phase system may comprise, as shown in FIG. 4:
an acquisition module 401, configured to acquire the total mass, total volume, and gas volume of fluids produced at the wellhead of a target well within a preset monitoring period, wherein the produced fluids are in an oil-water-gas three-phase mixed state;
an obtaining module 402, configured to obtain the emulsion ratio, emulsion volume coefficient, oil density, water density, and gas density of the fluids;
a first determining module 403, configured to determine the liquid-phase mass and liquid-phase volume of the fluids according to the total mass, total volume, gas volume, and gas density;
a calculating module 404, configured to calculate the oil mass fraction in the liquid phase of the fluids based on the liquid-phase mass, liquid-phase volume, emulsion ratio, emulsion volume coefficient, oil density, and water density;
a second determining module 405, configured to determine the water cut and oil-gas ratio of the fluids according to the oil mass fraction in the liquid phase of the fluids.

In one embodiment, the calculating module 404 is specifically configured to calculate the oil mass fraction in the liquid phase of the fluids according to the following formula:

$${V}_{l} = ( 1 - r ) \frac{( 1 - α ) {M}_{l}}{{ρ}_{w}} + δ [ \frac{α {M}_{l}}{{ρ}_{o}} + r \frac{( 1 - α ) {M}_{l}}{{ρ}_{w}} ]$$

wherein α represents the oil mass fraction, r represents the emulsion ratio, δ represents the emulsion volume coefficient, Mₗ represents the liquid-phase mass, ρ_{w} represents the water density, ρₒ represents the oil density, and Vₗ represents the liquid-phase volume.

In one embodiment, the calculating module 404 is specifically configured to calculate the oil mass fraction in the liquid phase of the fluids according to the following formula:

$${V}_{l} = ( 1 - r ) \frac{( 1 - α ) {M}_{l}}{{ρ}_{w}} + ( 1 - s ) \frac{α {M}_{l}}{{ρ}_{o}} δ [ s \frac{α {M}_{l}}{{ρ}_{o}} + r \frac{( 1 - α ) {M}_{l}}{{ρ}_{w}} ]$$

wherein α represents the oil mass fraction, r represents the emulsion ratio, δ represents the emulsion volume coefficient, Mₗ represents the liquid-phase mass, ρ_{w} represents the water density, ρₒ represents the oil density, Vₗ represents the liquid-phase volume, and s represents the proportion of the oil phase that can be emulsified.

In one embodiment, the second determining module 405 is specifically configured to calculate the water cut according to the following formula:

$${f}_{w} = \frac{( 1 - α ) {ρ}_{o}}{α {ρ}_{w} + ( 1 - α ) {ρ}_{o}}$$

wherein f_{w} represents the water cut, α represents the oil mass fraction, ρ_{w} represents the water density, and ρₒ represents the oil density.

In one embodiment, the second determining module 405 is specifically configured to calculate the gas-oil ratio according to the following formula:

$$GOR = \frac{{v}_{g} {ρ}_{0}}{α ( {M}_{t o t a l} - {ρ}_{g} {v}_{g} )}$$

wherein GOR represents the oil-gas ratio, ν_{g} represents the gas volume, ρ_{w} represents the water density, ρₒ represents the oil density, and Mₜₒₜₐₗ represents the total mass.

In one embodiment, after determining the gas-oil ratio of the fluids according to the oil mass fraction in the liquid phase of the fluids, the second determining module 405 may further convert the gas volume into a standard volume under standard temperature and pressure, and calculate the gas-oil ratio under standard conditions according to the following formula:

$${GOR}_{s t d} = \frac{{V}_{g}^{s t d}}{{V}_{o}} wherein {V}_{g}^{s t d}$$

represents the standard volume under standard temperature and pressure, and Vₒ represents the oil phase volume.

In one embodiment, the acquisition module 402 may specifically acquire the temperature and pressure of the target wellhead during a preset monitoring period; retrieve a pre-established data reference table, wherein the data reference table records the emulsion ratio, emulsion volume coefficient, oil density, water density, and gas density of the target well fluids under different temperature and pressure conditions, which are determined by external experiments; according to the measured temperature and pressure, search the data reference table for the corresponding emulsion ratio, emulsion volume coefficient, oil density, water density, and gas density; and use the retrieved values as the emulsion ratio, emulsion volume coefficient, oil density, water density, and gas density of the fluids.

In one embodiment, after determining the water cut and gas-oil ratio of the fluids according to the oil mass fraction in the liquid phase of the fluids, the determination of the water cut and gas-oil ratio of the three-phase system may further include determining the required separator pressure and the required demulsifier injection amount based on the water cut and gas-oil ratio; adjusting the separator pressure according to the determined pressure; and injecting the demulsifier according to the determined injection amount.

The embodiments of the present application further provide a specific embodiment of an electronic device capable of implementing all steps of the method for determining the water cut and gas-oil ratio of the three-phase system described above. The electronic device specifically includes: a processor, a memory, a communications interface, and a bus; wherein the processor, memory, and communications interface complete mutual communication through the bus; the processor is configured to call a computer program in the memory, and when the processor executes the computer program, all steps of the method for determining the water cut and gas-oil ratio of the three-phase system in the embodiments of the present application are implemented. For example, when the processor executes the computer program, the following steps are implemented:
Step 1: acquiring the total mass, total volume, and gas volume of the fluids produced at the target wellhead during a preset monitoring period, wherein the produced fluids are in an oil-water-gas three-phase mixed state;
Step 2: acquiring the emulsion ratio, emulsion volume coefficient, oil density, water density, and gas density of the fluids;
Step 3: determining the liquid-phase mass and liquid-phase volume of the fluids according to the total mass, total volume, gas volume, and gas density of the fluids;
Step 4: calculating the oil mass fraction in the liquid phase of the fluids according to the liquid-phase mass, liquid-phase volume, emulsion ratio, emulsion volume coefficient, oil density, and water density of the fluids;
Step 5: determining the water cut and gas-oil ratio of the fluids according to the oil mass fraction in the liquid phase of the fluids.

The embodiments of the present application further provide a computer-readable storage medium configured to implement all steps of the method for determining the water cut and gas-oil ratio of the three-phase system described above, wherein a computer program is stored on the computer-readable storage medium, and the computer program, when executed by a processor, implements all steps of the method for determining the water cut and gas-oil ratio of the three-phase system in the embodiments of the present application. For example, when the processor executes the computer program, the following steps are implemented:
Step 1: acquiring the total mass, total volume, and gas volume of the fluids produced at the target wellhead during a preset monitoring period, wherein the produced fluids are in an oil-water-gas three-phase mixed state;
Step 2: acquiring the emulsion ratio, emulsion volume coefficient, oil density, water density, and gas density of the fluids;
Step 3: determining the liquid-phase mass and liquid-phase volume of the fluids according to the total mass, total volume, gas volume, and gas density of the fluids;
Step 4: calculating the oil mass fraction in the liquid phase of the fluids according to the liquid-phase mass, liquid-phase volume, emulsion ratio, emulsion volume coefficient, oil density, and water density of the fluids;
Step 5: determining the water cut and gas-oil ratio of the fluids according to the oil mass fraction in the liquid phase of the fluids.

From the above description, it can be seen that the embodiments of the present application, by combining the total mass, total volume, and gas volume of the fluids produced at the target wellhead during a preset monitoring period with the acquired emulsion ratio, emulsion volume coefficient, oil density, water density, and gas density of the fluids, calculate the oil mass fraction in the liquid phase of the fluids, thereby further determining the water cut and gas-oil ratio of the fluids. That is, by introducing the emulsion ratio and the emulsion volume coefficient of the fluids into the calculation of the water cut and gas-oil ratio, the problem of low calculation accuracy of water cut and gas-oil ratio in the prior art is solved, achieving the technical effect of efficiently and accurately determining the water cut and gas-oil ratio.

The embodiments in this specification are described in a progressive manner, and the same or similar parts among the embodiments may be referred to each other, while the emphasis of each embodiment is on the differences from other embodiments. In particular, for hardware-plus-program embodiments, since they are basically similar to method embodiments, the description is relatively simple, and relevant parts may be referred to the description of the method embodiments.

The above description is only for the embodiments of the present specification and is not intended to limit the embodiments of the present specification. Other embodiments fall within the scope of the appended claims. In some cases, the actions or steps recited in the claims may be performed in an order different from that of the embodiments and still achieve the desired results. In addition, the processes depicted in the drawings do not necessarily require the specific order or sequential order shown to achieve the desired results. In some embodiments, multitasking and parallel processing are also possible or advantageous.

Although the embodiments of the present application provide method operation steps as described in the embodiments or flowcharts, more or fewer operation steps may be included based on conventional or non-inventive work. The sequence of steps listed in the embodiments is merely one way among many possible execution orders and does not represent the only execution order. In actual device or client execution, the method may be executed sequentially as illustrated in the embodiments or drawings, or executed in parallel (for example, in an environment of parallel processors or multithreaded processing).

Although the embodiments of the present specification provide method operation steps as described in the embodiments or flowcharts, more or fewer operation steps may be included based on conventional or non-inventive means. The sequence of steps listed in the embodiments is merely one way among many possible execution orders and does not represent the only execution order. In actual device or terminal execution, the method may be executed sequentially as illustrated in the embodiments or drawings, or executed in parallel (for example, in an environment of parallel processors, multithreaded processing, or even distributed data processing). The term “comprising,” “including,” or any other variation thereof is intended to cover a non-exclusive inclusion, such that a process, method, product, or device comprising a series of elements not only includes those elements but may also include other elements not expressly listed, or may further include inherent elements of the process, method, product, or device. Without further limitation, a process, method, product, or device comprising the stated elements does not exclude the presence of additional identical or equivalent elements.

For convenience of description, the above apparatus is described by function in terms of various modules. Of course, in implementing the embodiments of this specification, the functions of the various modules may be implemented in the same or multiple pieces of software and/or hardware, or the modules that realize the same function may be implemented by a combination of multiple sub-modules or sub-units. The described apparatus embodiments are merely illustrative. For example, the division of units is only one logical functional division, and in actual implementation there may be other divisions. For example, multiple units or components may be combined or integrated into another system, or some features may be omitted or not performed. Additionally, the couplings or direct couplings or communication connections shown or discussed may be indirect couplings or communication connections through some interfaces, devices, or units, and may be electrical, mechanical, or other forms.

Those skilled in the art will also understand that, in addition to implementing the controller in the form of pure computer-readable program code, it is entirely possible to achieve the same function by logically programming the method steps so that the controller is implemented in the form of logic gates, switches, application-specific integrated circuits, programmable logic controllers, and embedded microcontrollers. Therefore, such a controller may be considered a hardware component, and the devices included therein for realizing various functions may also be regarded as structures within the hardware component. Alternatively, the devices for realizing various functions may even be regarded as both software modules implementing the methods and structures within the hardware component.

The present application is described with reference to flowcharts and/or block diagrams of methods, devices (systems), and computer program products according to embodiments of the present application. It should be understood that each process and/or block in the flowcharts and/or block diagrams can be implemented by computer program instructions. These computer program instructions may be provided to a general-purpose computer, special-purpose computer, embedded processor, or other programmable data processing apparatus to produce a machine, such that the instructions executed by the processor of the computer or other programmable data processing apparatus create means for implementing the functions specified in one or more processes or blocks in the flowcharts and/or block diagrams.

Those skilled in the art should understand that the embodiments of this specification may be provided as methods, systems, or computer program products. Therefore, the embodiments of this specification may take the form of a purely hardware embodiment, a purely software embodiment, or an embodiment combining software and hardware. Moreover, the embodiments of this specification may take the form of a computer program product implemented on a computer-readable storage medium (including but not limited to disk storage, CD-ROM, optical storage, etc.) containing computer-usable program code.

The embodiments of this specification may be described in the general context of computer-executable instructions executed by a computer, such as program modules. Generally, program modules include routines, programs, objects, components, data structures, etc., that perform particular tasks or implement particular abstract data types. The embodiments of this specification may also be practiced in distributed computing environments, where tasks are performed by remote processing devices connected via a communication network. In distributed computing environments, program modules may be located in local and remote computer storage media including storage devices.

The embodiments in this specification are described in a progressive manner, and the same or similar parts among the embodiments may be referred to each other, while the emphasis of each embodiment is on the differences from other embodiments. In particular, for system embodiments, since they are basically similar to method embodiments, the description is relatively simple, and relevant parts may be referred to the description of the method embodiments. In the description of this specification, references to the terms “one embodiment,” “some embodiments,” “example,” “specific example,” or “some examples” mean that the specific features, structures, materials, or characteristics described in connection with the embodiment or example are included in at least one embodiment or example of the present specification. The illustrative expressions of the above terms do not necessarily refer to the same embodiment or example. Furthermore, the specific features, structures, materials, or characteristics described may be combined in any suitable manner in one or more embodiments or examples. In addition, without contradiction, those skilled in the art can combine and integrate different embodiments or examples and the features of different embodiments or examples described herein.

The above description is merely exemplary embodiments of the present specification and is not intended to limit the embodiments of the present specification. For those skilled in the art, the embodiments of this specification may be modified and changed in various ways. Any modification, equivalent replacement, or improvement made within the spirit and principles of the embodiments of this specification shall be included within the scope of the claims of the embodiments of this specification.

1-10. (canceled)

11. A method for determining water cut and gas-oil ratio of a three-phase system, characterized in that the method comprises: collecting, within a preset monitoring period, a total mass, a total volume, and a gas volume of a fluid produced at a wellhead of a target oil well, wherein the produced fluid is in an oil-water-gas three-phase mixed state; obtaining an emulsification ratio, an emulsion volume coefficient, an oil density, a water density, and a gas density of the fluid; determining a liquid-phase mass and a liquid-phase volume of the fluid according to the total mass, the total volume, the gas volume, and the gas density of the fluid; calculating an oil mass fraction within the liquid phase of the fluid according to the liquid-phase mass, the liquid-phase volume, the emulsification ratio, the emulsion volume coefficient, the oil density, and the water density of the fluid; determining a water cut and a gas-oil ratio of the fluid according to the oil mass fraction within the liquid phase of the fluid; wherein calculating the oil mass fraction within the liquid phase of the fluid according to the liquid-phase mass, the liquid-phase volume, the emulsification ratio, the emulsion volume coefficient, the oil density, and the water density comprises: calculating the oil mass fraction within the liquid phase of the fluid according to the following formula:
$${V}_{l} = ( 1 - r ) \frac{( 1 - α ) {M}_{l}}{{ρ}_{w}} + δ [ \frac{α {M}_{l}}{{ρ}_{o}} + r \frac{( 1 - α ) {M}_{l}}{{ρ}_{w}} ]$$
alternatively, calculating the oil mass fraction within the liquid phase of the fluid according to the following formula:
$${V}_{l} = ( 1 - r ) \frac{( 1 - α ) {M}_{l}}{{ρ}_{w}} + ( 1 - s ) \frac{α {M}_{l}}{{ρ}_{o}} δ [ s \frac{α {M}_{l}}{{ρ}_{o}} + r \frac{( 1 - α ) {M}_{l}}{{ρ}_{w}} ]$$
wherein α represents the oil mass fraction, r represents the emulsion ratio, δ represents the emulsion volume coefficient, Mₗ represents the liquid-phase mass, ρ_{w} represents the water density, ρₒ represents the oil density, Vₗ represents the liquid-phase volume, and s represents the proportion of the oil phase that can be emulsified.

12. The method according to claim 11, characterized in that determining the water cut of the fluids based on the oil mass fraction in the liquid phase of the fluids comprises: calculating the water cut according to the following formula:
$${f}_{w} = \frac{( 1 - α ) {ρ}_{o}}{α {ρ}_{w} + ( 1 - α ) {ρ}_{o}}$$
wherein f_{w} represents the water cut, α represents the oil mass fraction, ρ_{w} represents the water density, and ρₒ represents the oil density.

13. The method according to claim 11, characterized in that determining the oil-gas ratio of the fluids based on the oil mass fraction in the liquid phase of the fluids comprises: calculating the oil-gas ratio according to the following formula:
$$GOR = \frac{{v}_{g} {ρ}_{o}}{α ( {M}_{t o t a l} - {ρ}_{g} {v}_{g} )}$$
wherein GOR represents the oil-gas ratio, ν_{g} represents the gas volume, ρ_{w} represents the water density, ρₒ represents the oil density, and Mₜₒₜₐₗ represents the total mass.

14. The method according to claim 13, characterized in that after determining the oil-gas ratio of the fluids based on the oil mass fraction in the liquid phase of the fluids, the method further comprises: converting the gas volume into a standard volume under standard temperature and pressure; and calculating the oil-gas ratio under standard conditions according to the following formula:
$${GOR}_{s t d} = \frac{{V}_{g}^{s t d}}{{V}_{o}} wherein {V}_{g}^{s t d}$$
represents the standard volume under standard temperature and pressure, and Vₒ represents the oil phase volume.

15. The method according to claim 11, characterized in that obtaining the emulsification ratio, the emulsion volume coefficient, the oil density, the water density, and the gas density of the fluid comprises: collecting a temperature and a pressure at the wellhead of the target oil well within the preset monitoring period; retrieving a pre-established data lookup table, wherein the data lookup table records, as determined by external experiments, emulsification ratios, emulsion volume coefficients, oil densities, water densities, and gas densities of the fluid of the target oil well corresponding to different temperatures and pressures; searching, according to the collected temperature and pressure, the data lookup table to obtain an emulsification ratio, an emulsion volume coefficient, an oil density, a water density, and a gas density corresponding to the temperature and pressure; and using the obtained emulsification ratio, emulsion volume coefficient, oil density, water density, and gas density as the emulsification ratio, the emulsion volume coefficient, the oil density, the water density, and the gas density of the fluid.

16. The method according to any one of claims 11 to 15, characterized in that after determining the water cut and the gas-oil ratio of the fluid according to the oil mass fraction within the liquid phase of the fluid, the method further comprises: determining a required pressure of a separator and a required injection amount of an emulsifying agent according to the water cut and the gas-oil ratio; adjusting a pressure of the separator according to the determined required pressure of the separator; and injecting the emulsifying agent according to the determined required injection amount of the emulsifying agent.

17. An electronic device, comprising a processor and a memory configured to store processor-executable instructions, characterized in that the processor, when executing the instructions, implements the steps of the method according to any one of claims 11 to 16.

18. A computer-readable storage medium, on which computer program instructions are stored, characterized in that the computer program instructions, when executed by a processor, implement the steps of the method according to any one of claims 11 to 16.
